(19) United States
(12) Patent Application Publication
(10) Pub. No.: US 2024/0122957 A1
(43) Pub. Date: Apr. 18, 2024

(54) USE OF NICOTINAMIDE MONONUCLEOTIDE (NMN) FOR THE PREVENTION AND/OR TREATMENT OF RHEUMATOID ARTHRITIS, AND CORRESPONDING COMPOSITIONS

(71) Applicant: NUVAMID SA, Epalinges (CH)

(72) Inventors: Guillaume BERMOND, MARSEILLE (FR); Laurent GARCON, SAUSSET LES PINS (FR)

(73) Assignee: NUVAMID SA, Epalinges (CH)

(21) Appl. No.: 17/768,575

(22) PCT Filed: Oct. 15, 2020
(86) PCT No.: PCT/EP2020/079014

§ 371 Date: Apr. 13, 2022

(30) Foreign Application Priority Data
Oct. 18, 2019 (FR) FR1911696

(51) Int. Cl. A61K 31/706; A61K 45/06; A61P 19/02

(52) U.S. Cl. CPC A61K 31/706; A61K 45/06; A61P 19/02

(57) ABSTRACT

The invention concerns nicotinamide mononucleotide, a pharmaceutically acceptable derivative, or a pharmaceutically acceptable salt thereof, for use in the prevention and/or treatment of rheumatoid arthritis, as well as compositions comprising it.

### FIELD OF THE INVENTION

This invention concerns the use of nicotinamide mononucleotide (NMN), a pharmaceutically acceptable derivative, or a pharmaceutically acceptable salt thereof, as well as compositions comprising it, for the prevention and/or treatment of rheumatoid arthritis.

### PRIOR ART

A joint is an assembly, in particular, of two cartilage-covered ends of bones, as well as a synovial membrane enveloping this assembly. The role of the synovial membrane is to facilitate joint movements by secreting a lubricant: synovial fluid. Inflammatory rheumatism consists, in particular, of inflammation of the synovial membrane. An excessive amount of synovial fluid is then secreted, and the synovial membrane thickens abnormally. The soft tissues and osseous surfaces of the joint are then damaged. The joint becomes abnormally swollen and painful, hindering movement.

Rheumatoid arthritis is a chronic inflammatory rheumatism that most commonly affects the hands, writs, and knees, but may also extend to other joints. Arthritides are sometimes associated with the presence of rheumatoid factor in the patient's blood, and may result in irreversible joint damage.

Arthritis treatments comprise symptomatic treatment to relieve inflammatory eruptions on the one hand and treatment of the underlying disease on the other.

Symptomatic treatment primarily comprises administering analgesics to reduce pain, NSAIDs (non-steroidal anti-inflammatory drugs), as well as cortisone or derivatives thereof to reduce inflammation. However, the chronic administration of these drugs causes damage, inter alia, to the stomach, liver, and kidneys. Moreover, their efficacy decreases over time, requiring increases in dosage or the use of more aggressive drugs that often cause greater side-effects. Additionally, the chronic use of cortisone derivatives causes, inter alia, bone fragility, neuropsychiatric effects, muscle wasting, and reduced immunity, leaving the patient vulnerable to infections.

Treatments of the underlying condition most commonly comprise administering methotrexate, an anti-cancer agent that has been successfully used to prevent and reduce the number of inflammatory eruptions. However, this drug has numerous side-effects, such as fever, anaemia, respiratory distress, risks of teratogenicity, and bone marrow toxicity, amongst other risks. As such, it is not well tolerated by all patients. Other drugs are used in lieu of or in conjunction with methotrexate, e.g. certain anti-malarials and inhibitors of TNF (Tumour Necrosis Factor), a protein implicated in inflammatory processes. However, none of these drugs is free of side-effects. In particular, there is a risk of weakening the immune system, as well as substantial toxicity to the patient's vital organs, such as the liver and kidneys.

Furthermore, rheumatoid arthritis (RA) stiffens and deforms joints. In addition to the aesthetic aspect, which is difficult to tolerate, everyday actions become more and more difficult to patients. Thus, patients ultimately need to change their daily routines, leave their jobs, and depend on assistance from others, which, inter alia, involves considerable costs to health systems.

Thus, there is a need to develop novel compositions for the treatment and/or prevention of RA that reduce the disadvantageous of the prior art.

### SUMMARY OF THE INVENTION

These objectives are met by the invention as described infra.

This invention concerns nicotinamide mononucleotide (NMN), a pharmaceutically acceptable derivative, or a pharmaceutically acceptable salt thereof, for use in the prevention and/or treatment of rheumatoid arthritis.

Advantageously, the pharmaceutically acceptable derivative of the NMN is dihydronicotinamide mononucleotide ('NMN—H′), the compound of formula (I):

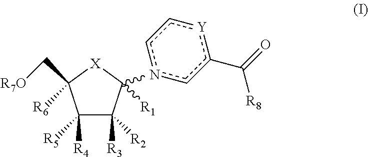

or a pharmaceutically acceptable stereoisomer, salt, hydrate, solvate, or crystal thereof, wherein
X is selected from O, CH₂, S, Se, CHF, CF₂, C═CH₂;
R₁ is selected from H, azido, cyano, (C₁-C₈) alkyl, (C₁-C₈) thioalkyl, (C₁-C₈) heteroalkyl, and OR; wherein R is selected from H and (C₁-C₈) alkyl;
R₂, R₃, R₄, and R₅ are selected independently of one another from H, halogen, azido, cyano, hydroxyl, (C₁-C₁₂) alkyl, (C₁-C₁₂) thioalkyl, (C₁-C₁₂) heteroalkyl, (C₁-C₁₂) haloalkyl, and OR; wherein R is selected from H, (C₁-C₁₂) alkyl, C(O)(C₁-C₁₂) alkyl, C(O)NH(C₁-C₁₂) alkyl, C(O)O(C₁-C₁₂) alkyl, C(O) aryl, C(O)(C₁-C₁₂) alkylaryl, C(O)NH(C₁-C₁₂) alkylaryl, C(O)O(C₁-C₁₂) alkylaryl, and C(O)CHR_{AA}NH₂; wherein R_{AA} is a side chain selected from the proteinogenic amino acids;
R₆ is selected from H, azido, cyano, (C₁-C₈) alkyl, (C₁-C₈) thioalkyl, (C₁-C₈) heteroalkyl, OR; wherein R is selected from H and (C₁-C₈) alkyl;
R₇ is selected from P(O)R₉R₁₀ and P(S)R₉R₁₀; wherein
R₉ and R₁₀ are selected independently of one another from OH, OR₁₁, NHR₁₃, NR₁₃R₁₄, (C₁-C₈) alkyl, (C₂-C₈) alkenyl, (C₂-C₈) alkynyl, (C₃-C₁₀) cycloalkyl, (C₅-C₁₂) aryl, (C₁-C₈) alkylaryl, (C₁-C₈) arylalkyl, (C₁-C₈) heteroalkyl, (C₁-C₈) heterocycloalkyl, heteroaryl, and NHCHR_{A}R_{A′}C(O)R₁₂; wherein:
R₁₁ is selected from a (C₁-C₁₀) alkyl, (C₃-C₁₀) cycloalkyl, (C₅-C₁₈) aryl, (C₁-C₁₀) alkylaryl, substituted (C₅-C₁₂) aryl, (C₁-C₁₀) heteroalkyl, (C₃-C₁₀) heterocycloalkyl, (C₁-C₁₀ haloalkyl, heteroaryl, —(CH₂)ₙC(O)(C₁-C₁₅) alkyl, —(CH₂)ₙOC(O)(C₁-C₁₅) alkyl, —(CH₂)ₙOC(O)O(C₁-C₁₅) alkyl, —(CH₂)ₙSC(O)(C₁-C₁₅) alkyl, —(CH₂)ₙC(O)O(C₁-C₁₅) alkyl, and —(CH₂)ₙC(O)O(C₁-C₁₅)alkylaryl group; wherein n is an integer from 1-8; P(O)(OH)OP(O)(OH)₂. halogen, nitro, cyano, C₁-C₆ alkoxy, C₁-C₆ haloalkoxy, —N(R₁₁ₐ)₂, C₁-C₆ acylamino, —COR_{11b}, —O COR_{11b}; NHSO₂(C₁-C₆ alkyl), —SO₂N(R₁₁ₐ)₂ SO₂; wherein each R₁₁ₐ is independently selected from H and (C₁-C₆) alkyl, and R_{11b} is independently selected from OH, C₁-C₆ alkoxy, NH₂, NH(C₁-C₆ alkyl), and N(C₁-C₆ alkyl)₂;
R₁₂ is selected from H, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈ aryl, C₁-C₄ alkylaryl, and C₅-C₁₂ heteroaryl; wherein the aryl or heteroaryl groups are optionally substituted with one or two groups selected from halogen, trifluoromethyl, C₁-C₆ alkyl, C₁-C₆ alkoxy, and cyano; and
R_{A} and R_{A′} are independently selected from H, (C₁-C₁₀) alkyl, (C₂-C₁₀) alkenyl, (C₂-C₁₀) alkynyl, (C₃-C₁₀) cycloalkyl, (C₁-C₁₀) thioalkyl, (C₁-C₁₀) hydroxylalkyl, (C₁-C₁₀) alkylaryl, and (C₅-C₁₂) aryl, (C₃-C₁₀) heterocycloalkyl, heteroaryl, —(CH₂)₃NHC(═NH)NH₂, (1H-indol-3-yl) methyl, (1H-imidazol-4-yl) methyl, and a side chain selected from a proteinogenic or non-proteinogenic amino acid; wherein the aryl groups are optionally substituted with a group selected from hydroxyl, (C₁-C₁₀) alkyl, (C₁-C₆) alkoxy, halogen, nitro, and cyano; or
R₉ and R₁₀ form, together with the phosphorus atoms to which they are attached, a 6-membered cycle, wherein —R₉—R₁₀— is —CH₂—CH₂—CHR—; wherein R is selected from a (C₅-C₆) aryl, and a (C₅-C₆) heteroaryl group; wherein the aryl or heteroaryl groups are optionally substituted with halogen, trifluoromethyl, C₁-C₆ alkyl, (C₁-C₆) alkoxy, and cyano; or
   R₉ and R₁₀ form, together with the phosphorus atoms to which they are attached, a 6-membered cycle, wherein —R₉—R₁₀— is —O—CH₂—CH₂—CHR—O—; wherein R is selected from a (C₅-C₆) aryl, and a (C₅-C₆) heteroaryl group; wherein the aryl or heteroaryl groups are optionally substituted with halogen, trifluoromethyl, (C₁-C₆) alkyl, (C₁-C₆) alkoxy, and cyano;
R₈ is selected from H, OR, NHR₁₃, NR₁₃R₁₄, NH—NHR₁₃, SH, CN, N₃, and halogen; wherein R₁₃ and R₁₄ selected, independently of one another, from H, (C₁-C₈) alkyl, (C₁-C₈) alkylaryl, and —CR_{B}R_{C}—C(O)—OR_{D}; wherein R_{B} and R_{C} are independently a hydrogen atom, (C₁-C₆) alkyl, (C₁-C₆) alkoxy, benzyl, indolyl, or imidazolyl; wherein the (C₁-C₆) alkyl and the (C₁-C₆) may, optionally and independently of one another, be substituted by one or more halogen, amino, amido, guanidyl, hydroxyl, thiol, or carboxyl groups; and the benzyl group is optionally substituted by one or more halogen or hydroxyl groups; or R_{B} and R_{C}, together with the carbon atom to which they are attached, form a C₃-C₆ cycloalkyl group optionally substituted with one or more halogen, amino, amido, guanidyl, hydroxyl, thiol, and carboxyl; and R_{D} is hydrogen, (C₁-C₆) alkyl, (C₂-C₆) alkenyl, (C₂-C₆) alkynyl, or (C₃-C₆) cycloalkyl;
Y is selected from CH, CH₂, C(CH₃)₂, and CCH₃;
[symbol] is a single or double bond depending on Y; and
[symbol] is the alpha or beta anomer depending on the position of R₁
or a pharmaceutically acceptable stereoisomer, salt, hydrate, solvate, or crystal thereof, or a combination thereof.

In a first preferred embodiment, the pharmaceutically acceptable derivative is the compound of formula (I).

In one variant of the first embodiment, X is oxygen.

In one variant of the first embodiment, R₁ and R₆ each, independently of one another, are hydrogen.

In one variant of the first embodiment, R₂, R₃, R₄, and R₅ each, independently of one another, are hydrogen or OH.

In one variant of the first embodiment, Y is CH.

In one variant of the first embodiment, Y is CH₂.

In one variant of the first embodiment, R₇ is P(O)(OH)₂.

In one variant of the first embodiment,

X is oxygen; and/or
R₁ and R₆ are each independently hydrogen; and/or
R₂, R₃, R₄, and R₅ each independently are hydrogen, or R₂, R₃, R₄, and R₅ is independently OH; and/or
Y is CH or CH₂; and/or
R₇ is P(O)R₉R₁₀, wherein R₉ and R₁₀ are independently selected from OH, OR₁₁, NHR₁₃, NR₁₃R₁₄, C₁-C₈ alkyl, C₂-C₈ alkenyl, C₂-C₈ alkynyl, C₃-C₁₀ cycloalkyl, C₅-C₁₂ aryl, C₁-C₈ arylalkyl, C₁-C₈ alkylaryl, C₁-C₈ heteroalkyl, C₁-C₈ heterocycloalkyl, heteroaryl, and NHCR_{A}R_{A′}C(O)R₁₂.

In a particularly preferred variant of the first embodiment, the compound of the invention is selected from the following compounds:

TABLE 1

| Compound | |
|---|---|
| (anomer) | Structure |
| I-A (Beta) | 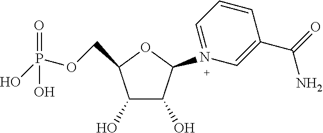 |
| I-B (beta) | 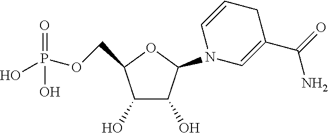 [SMILES: NC(=O)C1=CN([C@@H]2O[C@H](COP(=O)(O)O)[C@@H](O)[C@H]2O)C=CC1] |

In a preferred embodiment, the pharmaceutically acceptable derivative is NMN—H:

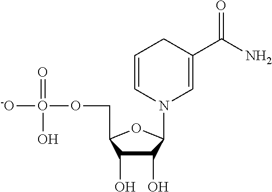

Advantageously, NMN or a pharmaceutically acceptable derivative or salt thereof may be used in an amount between 0.01 mg/kg/d and 1000 mg/kg/d, preferably between 1 mg/kg/d and 100 mg/kg/d, more preferably between 5 mg/kg/d and 50 mg/kg/d, even more preferably between 10 mg/kg/d and 20 mg/kg/d.

Advantageously, NMN or a pharmaceutically acceptable derivative or salt thereof may be administered orally, intraocularly, sublingually, intravenously, intraarterially, intramuscularly, intraarticularly, subcutaneously, transcutaneously, vaginally, peridurally, intravesically, rectally, or by inhalation.

In a preferred embodiment, NMN or a pharmaceutically acceptable derivative or salt thereof may be administered orally or by injection.

In a preferred embodiment, NMN or a pharmaceutically acceptable derivative or salt thereof may be administered orally, preferably in the form of a sublingual tablet or a gastroresistant capsule.

In an alternative preferred embodiment, NMN or a pharmaceutically acceptable derivative or salt thereof may be administered by injection, preferably intraarticular injection.

Advantageously, NMN or a pharmaceutically acceptable derivative or salt thereof may be used in the treatment and/or prevention of RA in mammals, preferably humans.

Advantageously NMN or a pharmaceutically acceptable derivative or salt thereof may be used in combination with at least one other therapeutic.

Advantageously, the at least one therapeutic may be an analgesic, an NSAID, cortisone, a cortisone derivative, an immunosuppressant, an immunomodulator, a T-lymphocyte inhibitor, a B-lymphocyte inhibitor, a synthetic antimalarial, an anti-TNF, an enzymatic Janus kinase inhibitor, an anti-interleukin, and combinations thereof.

Advantageously, the analgesic may be selected from paracetamol, aspirin, codeine, dihydrocodeine, tramadol, morphine, buprenorphin, fentanyl, hydromorphone, nalbuphine, oxycodone, pethidine, and combinations thereof.

Advantageously, the NSAID may be selected from ibuprofen, ketoprofen, naproxen, alminoprofen, aceclofenac, mefenamic acid, niflumic acid, tiaprofenic acid, celecoxib, dexketoprofen, diclofenac, etodolac, etoricoxib, fenoprofen, flurbiprofen, indomethacin, meloxicam, nabumetone, piroxicam, sulindac, tenoxicam, and combinations thereof.

Advantageously, the cortisone derivative may be selected from betamethasone, ciprofloxacin, cortivazol, dexamethasone, fludrocortisone, methylprednisolone, prednisolone, triamcinolone, and combinations thereof.

Advantageously, the immunosuppressant may be selected from azathioprine, cyclophosphamide, chlorambucil, cyclosporine, methotrexate, and combinations thereof.

In a preferred embodiment, the immunosuppressant may be methotrexate or cyclosporine, more preferably methotrexate.

Advantageously, the immunomodulator may be selected from leflunomide, sulphasalazine, and combinations thereof.

Advantageously, the synthetic antimalarial may be selected from chloroquine, hydroxychloroquine, and combinations thereof.

Advantageously, the anti-TNF agent may be selected from infliximab, etanercept, adalimumab, certolizumab, golimumab, and combinations thereof.

Advantageously, the enzymatic Janus kinase inhibitor may be tofacitinib.

Advantageously, the anti-interleukin may be selected from anti-interleukin 1, anti-interleukin 6, anti-interleukin 12, anti-interleukin 17, anti-interleukin 23, and combinations thereof.

Advantageously, the anti-interleukin 1 may be anakinra.

Advantageously, the anti-interleukin 6 may be tocilizumab or sarilumab

Advantageously, the B-lymphocyte inhibitor may be rituximab.

Advantageously, the T-lymphocyte inhibitor may be abatacept.

Advantageously, the interleukin 12 inhibitor may be ustekinumab.

Advantageously, the interleukin 17 inhibitor may be selected from ixekizumab and secukinumab.

Advantageously, the interleukin 23 inhibitor may be selected from ustekinumab and guselkumab.

Preferably, NMN or a pharmaceutically acceptable derivative or salt thereof is not used in combination with a compound selected from folate, S-adenosyl-L-methionine (SAM), astaxanthine, berberine, pterostilbene, resveratrol, metformin, vofloxacin, and combinations thereof.

This invention also concerns a composition comprising nicotinamide mononucleotide (NMN) or a pharmaceutically acceptable derivative or salt thereof and at least one pharmaceutically acceptable excipient for use in the prevention and/or treatment of RA.

Advantageously, the composition according to the invention may be provided in the form of a tablet, capsule, sachet, granulate, soft capsule, lyophilisate, suspension, gel, syrup, solution, water-in-oil emulsion, oil-in-water emulsion, oil, cream, milk, spray, ointment, ampule, suppository, eye drops, vaginal ovule, vaginal capsule, liquid for inhalation, dry powder inhaler, pressurised metered-dose inhaler.

Advantageously, the composition according to the invention may be a pharmaceutical composition.

Advantageously, the composition according to the invention may be a dietary supplement.

Advantageously, the composition according to the invention may be administered orally, intraocularly, sublingually, intravenously, intramuscularly, intraarticularly, subcutaneously, transcutaneously, vaginally, peridurally, intravesically, rectally, or by inhalation.

Advantageously, the composition according to the invention may be provided in a fixed unit-dose form.

Advantageously, the composition according to the invention may be administered orally or by injection.

In a preferred embodiment, the composition comprising NMN or a pharmaceutically acceptable derivative or salt thereof may be administered orally, preferably in the form of a sublingual tablet or a gastroresistant capsule.

In another preferred embodiment, the NMN or a pharmaceutically acceptable derivative or salt thereof may be administered by injection, preferably intraarticular injection.

In a preferred embodiment, the composition according to the invention may further comprise at least one additional therapeutic as defined supra for use in the prevention and/or treatment of RA as described supra.

Preferably, the composition according to the invention does not comprise a compound selected from folate, s-adenosyl-Lmethionine (SAM), astaxanthine, berberine, pterostilbene, resveratrol, metformin, vofloxacin, and combinations thereof.

### DEFINITIONS

In this invention, the following terms have the following meanings.

Unless otherwise indicated, the nomenclature of the substituents that are not explicitly defined in this invention is obtained by naming the terminal part of the functionality followed by the adjacent functionality in the direction of the point of attachment.

‘Alkyl’ on its own or as part of another substituent refers to a hydrocarbyl radical having the formula CnH2n+1, wherein n is a number greater than or equal to 1. In general, the alkyl groups of this invention comprise 1-12 carbon atoms, preferably 1-8 carbon atoms, more preferably 1-6 carbon atoms, even more preferably 1-2 carbon atoms. Alkyl groups may be linear or branched, and may be substituted as indicated in this invention. Alkyls suitable for the implementation of the invention may be selected from methyl, ethyl, n-propyl, i-propyl, n-butyl, i-butyl, s-butyl, and t-butyl, pentyl and isomers thereof such as n-pentyl and iso-pentyl, hexyl and isomers thereof such as n-hexyl and iso-hexyl, heptyl and isomers thereof (e.g. n-heptyl, iso-heptyl), octyl and isomers thereof (e.g. n-octyl, iso-octyl), nonyl and isomers thereof (e.g. n-nonyl, iso-nonyl), decyl and isomers thereof (e.g. n-decyl, iso-decyl), undecyl and isomers thereof, dodecyl and isomers thereof. Preferably, alkyl groups may be selected from methyl, ethyl, n-propyl, i-propyl, n-butyl, i-butyl, s-butyl, t-butyl, n-pentyl, n-hexyl, n-heptyl, n-octyl, n-nonyl, and n-decyl. Saturated and branched alkyl groups may be selected, without limitation, from isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, and 3,3-diethylhexyl. Preferred are the following alkyl groups: methyl, ethyl, n-propyl, i-propyl, n-butyl, i-butyl, s-butyl, and t-butyl. Cx-Cy alkyls refer to alkyl groups comprising x-y carbon atoms.

When the suffix ‘ene’ (‘alkylene’) is used in conjunction with an alkyl group, this means that the alkyl group as defined herein has two single bonds as points of attachment to other groups. The term ‘alkylene’ includes methylene, ethylene, methylmethylene, propylene, ethylethylene, and 1,2-dimethylethylene.

As used herein, the term ‘alkenyl’ refers to an unsaturated hydrocarbyl group that may be linear or ramified and comprises one or more carbon-carbon double bonds.

Suitable alkenyl groups comprise between 2 and 12 carbon atoms, preferably between 2 and 8 carbon atoms, and even more preferably between 2 and 6 carbon atoms. Examples of alkenyl groups include ethenyl, 2-propenyl, 2-butenyl, 3-butenyl, 2-pentenyl and isomers thereof, 2-hexenyl and isomers thereof, 2,4-pentadienyl, and similar groups.

As used herein, the term ‘alkynyl’ refers to a class of monovalent unsaturated hydrocarbyl groups, in which the unsaturation arises from the presence of one or more carbon-carbon triple bonds. Generally, and preferably, alkynyl groups have the same number of carbon atoms as described supra for alkenyl groups. Examples of alkynyl groups include, without limitation, ethynyl, 2-propynyl, 2-butynyl, 3-butynyl, 2-pentynyl and isomers thereof, 2-hexynyl and isomers thereof, etc.

‘Alkoxy’ refers to an alkyl group as defined supra that is attached to another part by an oxygen atom. Examples of alkoxy groups comprise, inter alia, methoxy, isopropoxy, ethoxy, and tert-butoxy groups. Alkoxy groups may be optionally substituted by one or more substituents. Alkoxy groups included in the compounds of this invention may optionally be substituted with a solubilising group.

As used herein, ‘aryl’ refers to a polyunsaturated aromatic hydrocarbyl group having a single cycle (e.g. phenyl) or several aromatic cycles fused together (e.g. naphtyl) or covalently bonded, generally containing 5-18 atoms, preferably 5-12, more preferably 6-10, at least one of which cycles is aromatic. The aromatic cycle may optionally comprise one or two additional cycles (cycloalkyl, heterocyclyl, or heteroaryl) fused with it. ‘Aryl’ is also intended to include partially hydrogenated derivatives of the carbocyclic systems set forth herein. Examples of aryl include phenyl, biphenylyl, biphenylenyl, 5- or 6-tetralinyl, naphtalene-1- or -2-yl, 4-, 5-, 6 or 7-indenyl, 1- 2-, 3-, 4- or 5-acenaphtylenyl, 3-, 4- or 5-acenaphtenyl, 1- or 2-pentalenyl, 4- or 5-indanyl, 5-, 6-, 7- or 8-tetrahydronaphtyl, 1,2,3,4-tetrahydronaphtyl, 1,4-dihydronaphtyl, 1-, 2-, 3-, 4- or 5-pyrenyl.

If at least one carbon atom in an aryl group is replaced by a heteroatom, the resultant cycle is referred to herein as a ‘heteroaryl’ cycle.

‘Alkylaryl’ refers to an aryl group substituted with an alkyl group.

‘Amino acid’ refers to an alpha-amino carboxylic acid, i.e. a molecule comprising a carboxylic acid functional group and an amino functional group in the alpha position of the carboxylic acid group, e.g. a proteinogenic amino acid or a non-proteinogenic amino acid.

‘Proteinogenic amino acid’ refers to an amino acid incorporated into proteins in the translation of messenger RNA (mRNA) by ribosomes in living organisms, i.e. Alanine (ALA), Arginine (ARG), Asparagine (ASN), Aspartate (ASP), Cysteine (CYS), Glutamate (glutamic acid) (GLU), Glutamine (GLN), Glycine (GLY), Histidine (HIS), Isoleucine (ILE), leucine (LEU), Lysine (LYS), Methionine (MET), Phenylalanine (PHE), Proline (PRO), Pyrrolysine (PYL), Selenocysteine (SEL), Serine (SER), Threonine (THR), Tryptophane (TRP), Tyrosine (TYR), or Valine (VAL).

As used herein, ‘non-proteinogenic amino acid’ refers to an amino acid that is not naturally encoded or found in the genetic code of a living organism. Examples of non-proteinogenic amino acids include, without limitation, ornithine, citrulline, argininosuccinate, homoserine, homocysteine, cysteine-sulphinic acid, 2-aminomuconic acid, δ-aminolevulinic acid, β-alanine, cystathionine, γ-aminobutyrate, DOPA, 5-hydroxytryptophan, D-serine, ibotenic acid, α-aminobutyrate, 2-aminoisobutyrate, D-leucine, D-valine, D-alanine, or D-glutamate.

As used herein, the term ‘cycloalkyl’ is a cyclic alkyl group, i.e. a saturated or unsaturated monovalent hydrocarbyl group having 1 or 2 cyclic structures. The term ‘cycloalkyl’ includes monocyclic or bicyclic hydrocarbyl groups. Cycloalkyl groups may comprise 3 or more carbon atoms in the cycle and, generally, according to this invention, may comprise 3-10, more preferably 3-8 carbon atoms, and even more preferably 3-6 carbon atoms. Examples of cycloalkyl groups include, without limitation, cyclopropyl, cyclobutyl, cyclopentyl, cyclohexyl, with cyclopropyl being particularly preferred.

‘Pharmaceutically acceptable excipient’ refers to an inert vehicle or support used as a solvent or diluent in which the active ingredient is formulated and/or administered, and that does not produce any undesirable, allergic, or other reaction when administered to an animal, preferably a human. This includes all solvents, dispersion media, coatings, antibacterials and antifungals, isotonic agents, absorption retardants, and other similar ingredients. For administration to humans, the preparations must meet sterility, general safety, and purity standards imposed by regulatory bodies such as the FDA or the EMA. Within the meaning of the invention ‘pharmaceutically acceptable excipient’ includes all pharmaceutically acceptable excipients, as well as all pharmaceutically acceptable supports, diluents, and/or adjuvants.

‘Halogen’ or ‘halo’ means fluoro, chloro, bromo, or iodo. Preferred halo groups are fluoro and chloro.

‘Haloalkyl’, alone or in combination, refers to an alkyl radical within the meaning set forth supra, wherein one or more hydrogen atoms are replaced by a halogen as defined supra. Examples of such haloalkyl radicals include chloromethyl, 1-bromoethyl, fluoromethyl, difluoromethyl, trifluoromethyl, 1,1,1-trifluoroethyl, and similar radicals. Cx-Cy-haloalkyl and Cx-Cy-alkyl refer to alkyl groups comprising x-y carbon atoms. Preferred haloalkyl groups are difluoromethyl and trifluoromethyl.

‘Heteroalkyl’ refers to an alkyl group as defined supra, wherein one or more carbon atoms are replaced by a heteroatom selected from oxygen, nitrogen, and sulphur atoms. In heteroalkyl groups, the heteroatoms are bonded along the alkyl chain only to carbon atoms, i.e. each heteroatom is separated from every other heteroatom by at least one carbon atom. However, nitrogen and sulphur atoms may be optionally oxidised, and nitrogen heteroatoms may optionally be quarternised. A heteroalkyl is bonded to another group or molecule only by a carbon atom, i.e. the bonding atom is not selected from the heteroatoms included within the heteroalkyl group.

As used herein, the term ‘heteroaryl’, whether alone or as part of another group, refers, without limitation, to aromatic cycles having 5-12 carbon atoms or cyclic systems containing 1 or 2 cycles that are fused or covalently bonded, generally containing 5 or 6 atoms, at least one of which is aromatic, wherein one or more carbon atoms in one or more of these cycles are replaced by oxygen, nitrogen, and/or sulphur atoms, wherein the nitrogen and sulphur heteroatoms may optionally be oxidised and the nitrogen heteroatoms may optionally be quarternised. These cycles may be fused with an aryl, cycloalkyl, heteroaryl, or heterocyclyl cycle. Examples of such heteroaryl groups include, without limitation, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo [2,1-b] [1,3] thiazolyl, thieno [3,2-b] furanyl, thieno [3,2-b] thiophenyl, thieno [2,3-d] [I,3] thiazolyl, thieno [2,3-d] imidazolyl, tetrazolo [I,5-a] pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl, imidazo[I,2-a]pyridinyl, 6-oxo-pyridazin-1(6H)-yl, 2-oxopyridin-I(2H)-yl, 6-oxo-pyridazin-I(6H)-yl, 2-oxopyridin-I(2H)-yl, 1,3-benzodioxolyl, quinolinyl, isoquinolinyl, cinnolinyl, quinazolinyl, quinoxalinyl.

If at least one carbon atom in a cycloalkyl group is replaced by a heteroatom, the resultant cycle is referred to herein as ‘hetercycloalkyl’ or ‘heterocyclyl’.

As used herein, the terms cheterocyclyr, cheterocycloalkyr, or ‘heterocyclo’, alone or as part of another group, refer to non-aromatic cyclic groups that are totally saturated or partially unsaturated, (e.g. monocyclic with 3-7 members, bicyclic with 7-11 members, or containing a total of 3-10 cycle atoms) having at least one heteroatom in at least one cycle containing a carbon atom. Each cycle of the heterocyclic group containing a heteroatom may have 1, 2, 3, or 4 heteroatoms selected from nitrogen, oxygen, and/or sulphur atoms, wherein the nitrogen and sulphur heteroatoms may optionally be oxidized and the nitrogen heteroatoms may optionally be quaternised. Any carbon atom of the heterocyclic group may be substituted by an oxo (e.g. piperidone, pyrrolidinone). The heterocyclic group may be attached to any heteroatom or carbon atom of the cycle or cyclic system if the valence permits. The cycles of the multicyclic heterocycles may be fused, bridged, and/or bonded by one or more spiro atoms. Non-limiting examples of heterocyclic groups include oxetanyl, piperidinyl, azetidinyl, 2-imidazolinyl, pyrazolidinyl, imidazolidinyl, isoxazolinyl, oxazolidinyl, isoxazolidinyl, thiazolidinyl, isothiazolidinyl, piperidinyl, 3H-indolyl, indolinyl, isoindolinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, 2, 5-dioxim idazolidinyl, 2-oxopiperidinyl, 2-oxopyrrolodinyl, indolinyl, tetrahydropyranyl, tetrahydrofuranyl, tetrahydroquinolinyl, tetrahydroisoquinolin-1-yl, tetrahydroisoquinolin-2-yl, tetrahydroisoquinolin-3-yl, tetrahydroisoquinoleine-4-yl [sic], thiomorpholine-4-yl, thiomorpholine-4-ylsulph [sic] oxide, thiomorpholine-4-ylsulphone, 1,3-dioxolanyl, 1,4-oxathianyl, 1H-pyrrolizinyl, tetrahydro-I,I-dioxothiophenyl, N-formylpiperazinyl, and morpholine-4-yl.

As used herein, ‘precursor’ also refers to pharmaceutically acceptable derivatives of the compounds of formula (I), such as esters, the in vivo biotransformation project of which is the active drug. Precursors are characterised by increased bioavailability, and they are easily metabolised into active compounds in vivo. Precursors suited to the purposes of the invention include, without limitation, carboxylic esters, in particular alkylic esters, arylic esters, acyloxyalkylic esters and carboxylic esters of dioxolene; ascorbic acid esters.

‘Pharmaceutically acceptable’ means approved or eligible for approval by a regulatory body or registered in a recognised pharmacopoeia for use in animals, and more preferably in humans. This may be a substance that is not undesirable in biological or other terms, i.e. the substance may be administered to an individual without causing undesirable biological effects or deleterious interactions with one of the components of the composition in which it is contained. Preferably, a ‘pharmaceutically acceptable’ salt or excipient refers to any salt or excipient authorized by the European Pharmacopoeia ('Ph. Eur.') and the United States Pharmacopeia (‘USP’).

‘Active ingredient’ refers to a molecule or substance, the administration of which to a subject slows or stops the progression, worsening, or deterioration of one or more symptoms of a disease or condition, relieves the symptoms of a disease or condition, or cures a disease or condition. In one of these embodiments, the therapeutic ingredient is a small molecule, natural or synthetic. In another, the therapeutic ingredient is a biological molecule, e.g. an oligonucleotide, siRNA, miRNA, DNA fragment, aptamer, antibody, etc. ‘Pharmaceutically acceptable salts’ include acid and base addition salts of these salts. Suitable acid addition salts are formed from acids that form non-toxic salts. These include, for example, acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, chlorhydrate/chlorure, bromhydrate/bromure, hydroiodure/iodure, isethionate, lactate, malate, maleate, malonate, mesylate, methylsulphate, naphtylate, 2-napsylate, nicotinate, nitrate, orotate, oxalate, palmitate, pamoate, phosphate/hydrogenophosphate/dihydrogenophosphate, pyroglutamate, saccharate, stearate, succinate, tannate, tartrate, tosylate, trifluoroacetate, and xinofoate salts. Suitable basic salts are formed from bases that form non-toxic salts. Examples include aluminium, arginine, benzathine, calcium, choline, diethylamine, diolamine, glycine, lysine, magnesium, meglumine, olamine, potassium, sodium, tromethamine, 2-(diethylamino)ethanol, ethanolamine, morpholine, 4-(2-hydroxyethyl)morpholine, and zinc salts. Hemisalts of acids and bases may also be formed, e.g. hemisulphates and chemical calcium salts. Preferred pharmaceutically acceptable salts are chlorhydrate/chloride, bromide/hydrobromide, bisulphate/sulphate, nitrate, citrate, and acetate.

Pharmaceutically acceptable salts may be prepared by one or more of the following methods:

i. by reacting the compound with the desired acid;
ii. by reacting the compound with the desired base;
iii. by eliminating an acid- or base-labile protecting group of a suitable precursor of the compound or by opening the cycle of a suitable cyclic precursor, e.g. a lactone or lactam, using the desired acid; or
iv. by transforming a salt of the compound into another by reacting it with a suitable acid or by means of a suitable ion exchange column.

All these reactions are generally carried out in solution. The salt may precipitate from the solution and be collected by filtration, or it may be recovered by evaporating the solvent. The degree of ionisation of the salt may vary from completely ionised to nearly non-ionised.

‘Solvate’ is used herein to describe a molecular complex comprising the compound of the invention and one or more pharmaceutically acceptable solvent molecules, e.g. ethanol.

The term ‘substituent’ or ‘substituted’ means that a hydrogen radical on a compound or group is replaced by any desired group that is substantially stable in the reaction conditions in a non-protected form or when protected by a protecting group. Examples of preferred substituents include, without limitation, halogen (chloro, iodo, bromo, or fluoro); alkyl; alkenyl; alkynyl, as described supra; hydroxy; alkoxy; nitro; thiol; thioether; imine; cyano; amido; phosphonato; phosphine; carboxyl; thiocarbonyl; sulphonyl; sulphonamide; ketone; aldehyde; ester; oxygen (—O); haloalkyl (e.g. trifluoromethyl); cycloalkyl, which may be monocyclic or polycyclic, condensed or non-condensed (e.g., cyclopropyl, cyclobutyl, cyclopentyl, or cyclohexyl), or heterocycloalkyl, which may be monocyclic or polycyclic, condensed or non-condensed (e.g., pyrrolidinyl, piperidinyl, piperazinyl, morpholinyl, or thiazinyl), monocyclic or polycyclic, fused or unfused, aryl or heteroaryl (e.g., aryl, heteroaryl, pyrrolidinyl, piperidinyl, piperazinyl, morpholinyl, or thiazinyl), monocyclic or polycyclic, fused or unfused (e.g., aryl, heteroaryl, pyrrolidinyl, piperidinyl, piperazinyl, morpholinyl, or thiazinyl), phenyl, naphtyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidinyl, benzimidazolyl, benzothiophenyl, or benzofuranyl); amino (primary, secondary, or tertiary); CO₂CH₃; CONH₂; OCH₂CONH₂; NH₂; SO₂NH₂; OCHF₂; CF₃; OCF₃; and these groups may also be optionally substituted by a structure or fused annular bridge, e.g. —OCH₂O—. These substituents may also optionally be substituted with a substituent chosen from these groups. In certain representations, the term ‘substituent’ or the adjective ‘substituted’ refers to a substituent selected from the group consisting of alkyl, alkenyl, alkynyl, cycloalkyl, cycloalkenyl, heterocycloalkyl, aryl, heteroaryl, arylalkyl, heteroarylalkyl, haloalkyl, —C(O)NR₁₁R₁₂, —NR₁₃C(O)R₁₄, halo, —OR₁₃, cyano, nitro, un haloalkoxy, —C(O)R₁₃, —NR₁₁R₁₂, —SR₁₃, —C(O)OR₁₃, —OC(O)R₁₃, —NR₁₃C(O)NR₁₁R₁₂, —OC(O)NR₁₁R₁₂, —NR₁₃C(O)OR₁₄, —S(O)rR₁₃, —NR₁₃S(O)rR₁₄, —OS(O)rR₁₄, S(O)rNR₁₁R₁₂, —O, —S, and —N—R₁₃, wherein r is 1 or 2; R₁₁ and R₁₂, in each occurrence, are independently H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, or optionally substituted heteroarylalkyl; or R₁₁ and R₁₂, taken together with the nitrogen to which they are attached, are optionally substituted heterocycloalkyl or optionally substituted heteroaryl; and R13 and R14, in each occurrence, are independently H, optionally substituted alkyl, optionally substituted alkenyl, optionally substituted alkynyl, optionally substituted cycloalkyl, optionally substituted cycloalkenyl, optionally substituted heterocycloalkyl, optionally substituted aryl, optionally substituted heteroaryl, optionally substituted arylalkyl, or optionally substituted heteroarylalkyl. In certain variants, the term ‘substituent’ or the adjective ‘substituted’ refers to a solubilising group.

The term ‘administration’ or any variant thereof (e.g. ‘to administer’) means providing the active ingredient, alone or within a pharmaceutically acceptable composition, to a patient in need of treatment or prevention of the condition, symptom, or disease.

‘To treat’, ‘to care’, and ‘treatment’, as used herein, are intended to include relieving, attenuating, or eliminating a condition or disease and/or the associated symptoms.

‘To prevent’ and ‘prevention’, as used herein, refer to a method allowing for the appearance of a condition or disease and/or its associated symptoms to be delayed or prevented, for a patient to be prevented from contracting a condition or disease, or for a reduction in a patient's risk of contracting a condition or disease.

Asymmetrical carbon bonds may be represented here using an unbroken triangle ( [symbol] ) a dotted triangle ( [symbol] ), or a zigzag line ( [symbol] )

### DETAILED DESCRIPTION OF THE INVENTION

This invention concerns nicotinamide mononucleotide (NMN) or a pharmaceutically acceptable derivative or salt thereof for use in the prevention and/or treatment of rheumatoid arthritis (RA), as well as compositions comprising it.

Nicotinamide adenine dinucleotide (NAD) is a coenzyme present in all living cells. NAD exists in the cell either in its oxidised form, NAD+, or in the reduced form, NADH. The role of NAD is as a transporter of electrons involved in metabolic redox reactions. NAD is also involved numerous cellular processes, such as the ribosylation of ADP in post-translational protein modifications.

NAD may be synthesised de novo by the cell from amino acids such as tryptophan or aspartate. However, this synthesis is marginal, because the main synthesis pathway of NAD is that of salvage, by which the cell, and mainly the cell nucleus, recycles compounds to re-form NAD from precursors. NAD precursors include niacin, nicotinamide riboside, nicotinamide mononucleotide, and nicotinamide.

NMN is one of the compounds allowing for NAD synthesis via salvage, and has the following formula:

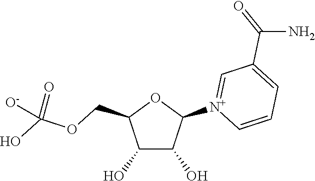

Indeed, the inventors have shown that the use of NMN or pharmaceutically acceptable salts and/or derivatives thereof, and of the composition according to the invention allows for an effect on joint swelling caused by RA that is comparable with the drugs currently used to treat this disease without having the same side effects. More precisely, the inventors have found that NMN was able to treat the inflammatory eruptions characteristic of RA by significantly reducing joint swelling, and did so with comparable efficacy to conventional treatments. Moreover, the chronic administration of NMN allows for prevention, or at least increasing the intervals between, these eruptions. In fact, when administered chronically between each eruption, NMN and compositions comprising it are able to reduce inflammation and thus to prevent, or at least increase the intervals between, RA eruptions.

Moreover, the use of NMN, a molecule that is naturally present in the body, has numerous advantages. In particular, NMN does not raise any tolerance issues for patients. Indeed, the use of NMN and the composition according to the invention does not induce any allergic reaction. Moreover, the use of NMN and the composition according to the invention does not cause the side-effects frequently encountered with conventional treatments.

In particular, NMN does not cause any physical or psychological dependency, unlike analgesics containing morphine or opium derivatives. Furthermore, NMN does not cause bone fragility or vulnerability to infection as has been observed with the chronic administration of cortisone or its derivatives. Thus, the use of NMN and the composition according to the invention to prevent and/or treat RA is safe for patients.

NMN and the composition according to the invention may be used both in children and adults. Indeed, NMN is well tolerated by children. In the context of the invention, a patient is considered a child if their age is less than 18, and an adult starting at age 18. As such, the invention is also of interest for treating RA in children.

In a particularly preferred embodiment, the NMN is in the form of a zwitterion. A ‘zwitterion’ is a chemical molecular species having opposite electrical charges that are generally located on non-adjacent atoms of the molecule.

The use of NMN or a pharmaceutically acceptable derivative or salt thereof, as well as compositions according to the invention comprising it primarily makes it possible to treat inflammation during RA eruptions by reducing the inflammation and, in particular, joint swelling. Thus, in the long term, it is possible to avoid joint deformity, or at least to reduce or delay joint deformity. Thus, the patient's joints are preserved.

By reducing inflation, and, in particular, swelling, in joints, as well as preventing inflammatory eruptions, it is also possible to reduce the pain related to the inflammation and to reduce joint stiffening. Thus, it is possible to avoid administering, or at least to reduce the frequency of administration and the dosage of, drugs used to combat the symptoms of inflammation, i.e. analgesics, NSAIDs, cortisone, and/or cortisone and derivatives thereof. This also makes it possible to avoid the administration of treatments for the underlying condition that are conventionally used to treat chronic inflammatory rheumatism, such as methotrexate, or at least to reduce their dosage.

By reducing the need for conventional therapies, or even replacing them, this invention thus makes it possible to avoid, or at least reduce, the use of conventional RA treatments, and thus to avoid, or at least reduce, the appearance of side-effects connected to these treatments.

In addition to the therapeutic aspect, therefore, the invention makes it possible to maintain patients' quality of life by making it easier for them to carry out everyday tasks, and possibly avoiding the need to end professional activities. Thus, the invention serves to maintain, or at least to avoid excessive deterioration of, patient quality of life.

### Use

According to this invention, NMN or pharmaceutically acceptable derivatives or salts thereof, as well as compositions comprising it, are used to prevent and/or treat RA. More precisely, they may be used acutely to treat an eruption of RA or chronically to reduce inflammation and increase the intervals between eruptions. In other words, NMN or a pharmaceutically acceptable derivative or salt thereof, as well as compositions comprising them, may be used preventively or curatively in order to reduce inflammation, and in particular, swelling, of joints.

NMN or pharmaceutically acceptable derivatives or salts thereof, and the compositions according to the invention may be administered in a therapeutically effective amount. In the context of the invention, a therapeutically effective amount of a composition means that the composition is administered to a patient in an amount sufficient to obtain the desired therapeutic effect.

NMN or a pharmaceutically acceptable precursor, derivative, or salt thereof is used in an amount between 0.01 mg/kg/d and 1000 mg/kg/d, preferably between 1 mg/kg/d and 100 mg/kg/d, more preferably between 5 mg/kg/d and 50 mg/kg/d, even more preferably between 10 mg/kg/d and 20 mg/kg/d. Persons skilled in the art are able to adapt the dose of NMN to be administered based on the age and weight of the patient.

A suitable dosage level may be approximately 0.01-250 mg/kg/d, approximately 0.05-100 mg/kg/d, or approximately 0.1 à 50 mg/kg/d. Within this range, the dose may be 0.05-0.5, 0.5-5, or 5-50 mg/kg/d. For oral administration, the compositions are preferably provided in the form of tablets containing 1.0-1000 mg of NMN or a pharmaceutically acceptable precursor, derivative, or salt thereof, in particular 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 mg of active ingredient for symptom-based adjustment of the dose of the patient to be treated. For example, the dosage may be between 100 mg/d and 5000 mg/d, preferably between 500 mg/d and 1000 mg/d. The compounds may be administered according to a regimen of 1-4 administrations per day, preferably once, twice, or thrice a day, preferably thrice a day. The duration of treatment depends, and is determined by, the treating physician. It may range from one day to one year or more, preferably from one week to three months, more preferably from two weeks to six weeks. However, it is understood that the specific dosage level and frequency, as well as the duration for a given patient may vary, and will depend on various factors, in particular the activity of the specific compound used, the metabolic stability and duration of action of the compound, the age, body weight, general condition, sex, diet, mode and time of administration, excretion rate, combination of drugs, and the individual being treated.

NMN or a pharmaceutically acceptable precursor, derivative, or salt thereof may be administered at a daily dose of 10 mg/kg, with a minimum of 50 mg/d and a maximum of 1000 mg/d.

NMN and the composition according to the invention may be administered one or more times a day. In particular, NMN et the composition according to the invention may be administered between 1 and 12 a day, preferably between 2 and 10 times a day, more preferably between 3 and 5 times a day.

### Mode of Administration and Galenic Form

NMN or a pharmaceutically acceptable derivative or salt thereof, or compositions comprising it, may be administered orally, intraocularly, sublingually, intravenously, intraarterially, intramuscularly, intraarticularly, subcutaneously, transcutaneously, vaginally, peridurally, intravesically, rectally, or by inhalation.

Depending on the intended mode of administration the composition according to the invention may be provided in the form of a tablet, capsule, sachet, granulate, soft capsule, lyophilisate, suspension, gel, syrup, solution, water-in-oil emulsion, oil-in-water emulsion, oil, cream, milk, spray, ointment, ampoule, suppository, eye drops, vaginal ovule, vaginal capsule, liquid for inhalation, dry powder inhaler, pressurised metered-dose inhaler.

In a preferred embodiment, NMN and the composition according to the invention are administered by injection, in particular subcutaneously, intravenously, or intraarticularly, preferably intraarticularly.

In another embodiment that is also preferred, NMN and the composition according to the invention are administered orally.

The oral form according to the invention may also be an immediate release form: such a galenic form allows for rapid absorption of NMN and reduced delay in onset of action. Galenic forms for immediate release include, inter alia, dispersible, orodispersible, and effervescent tablets, as well as oral lyophilisates.

Dispersible tablets are uncoated or film-coated tablets that may be dispersed in a liquid prior to administration in order to ensure homogeneous dispersion. The dispersible tablets usually disintegrate within three minutes once placed in water or a small quantity of breast milk.

An effervescent tablet is a tablet designed to fragment and dissolve rapidly in water or another liquid whilst releasing carbon dioxide (CO2). This release causes effervescence and the fragmentation of the tablet.

An orodispersible tablet is a dispersible tablet that is placed on the tongue. The active ingredient is then absorbed by the gastrointestinal mucosa.

‘Sublingual tablet’ refers to an oral lyophilisate that is placed under the tongue so that the active ingredient is absorbed by the sublingual mucosa, in particular by the lingual vein and artery.

The oral form according to the invention may also be a delayed release form: NMN is dissolved and absorbed in the intestines, thus limiting gastric irritation or the breakdown of fragile active ingredients at acidic pH. These are mostly gastroresistant forms, i.e. the tablets or granulates are coated in a polymer film that is insoluble in an acidic medium but permeable to water in an alkaline medium, or a lipid film that is broken down by the intestinal lipases.

‘Gastroresistant’ refers to a galenic form that does not dissolve in the stomach. Such galenic forms are for delayed release, i.e. they have a coating or a coating composition that is resistant to the acidic pH of the stomach (pH<2) in order to dissolve in the intestines. Whether a galenic form is gastroresistant is determined based on the test established by the European Pharmacopoeia. In brief, the gastroresistance of a capsule is measured in a disintegration medium of 0.1 M hydrochloric acid at 37° C. in a disintegration device. This environment mimics the physico-chemical conditions of the stomach. The capsules are incubated in this environment for 1 h. The capsule must not show any signs of disintegration or fissures that might result in loss of contents. Then, the capsule is incubated for 1 h in a phosphate buffer solution at pH 6.8 at 37° C.; this solution mimics the conditions of the intestinal environment in accordance with the recommendations of the European Pharmacopoeia. The capsule must be totally disintegrated within less than one hour.

The oral form according to the invention may also be a sustained and sequential release form: Sequential (release at precise time intervals) and sustained release forms (continuous release of active ingredient until exhaustion) spread out the release of the active ingredient over time in order to maintain an effective plasma concentration for a longer time in the patient's body. Such galenic forms allow for relief over a longer period of time, and make it possible to increase the interval between doses of the drug.

In a more preferred embodiment, NMN and the composition according to the invention are administered orally in the form of a gastroresistant capsule or a sublingual tablet.

The mode of administration and galenic form are determined by persons skilled in the art depending on the patient and the anatomic site to be treated. Reference is made to the most recent edition of Remington's Pharmaceutical Sciences.

### Therapeutic Combinations

Le NMN, a pharmaceutically acceptable derivative or salt thereof, and compositions comprising them may also be used in combination with at least one other therapeutic, in particular therapeutics used either acutely to treat RA eruptions, i.e. symptomatic treatments, or for treatment of the underlying disease process of RA .

A treatment for the underlying disease process is a treatment taken daily, on a chronic basis, in order to prevent or increase the intervals between crises.

Therapeutics that may be combined according to the invention include analgesics, NSAIDs, cortisone, a cortisone derivative, immunosuppressants, immunomodulators, T-lymphocyte inhibitors, B-lymphocyte inhibitors, synthetic antimalarials, anti-TNF agents, enzymatic Janus kinase inhibitors, anti-interleukin agents, and combinations thereof.

More specifically, analgesics, NSAIDs, and cortisone and derivatives thereof may be used to treat and relieve eruptions of inflammatory rheumatic disorders as symptomatic treatments for inflammatory eruptions.

The analgesic may be selected from paracetamol, aspirin, codeine, dihydrocodeine, tramadol, morphine, buprenorphin, fentanyl, hydromorphone, nalbuphine, oxycodone, pethidine, and combinations thereof.

The NSAID may be elected from ibuprofen, ketoprofen, naproxen, alminoprofen, aceclofenac, mefenamic acid, niflumic acid, tiaprofenic acid, celecoxib, dexketoprofen, diclofenac, etodolac, etoricoxib, fenoprofen, flurbiprofen, indomethacin, meloxicam, nabumetone, piroxicam, sulindac, tenoxicam, and combinations thereof.

The cortisone derivative may be selected from betamethasone, ciprofloxacin, cortivazol, dexamethasone, fludrocortisone, methylprednisolone, prednisolone, triamcinolone, and combinations thereof.

NMN, pharmaceutically acceptable derivatives or salts thereof, as well as compositions comprising them may also be administered in combination with a treatment of the underlying disease process of RA, such as an immunosuppressant, immunomodulator, T-lymphocyte inhibitor, B-lymphocyte inhibitor, synthetic antimalarial, anti-TNF agent, enzymatic Janus kinase inhibitor, anti-interleukin agent, or combinations thereof. The use of a treatment of the underlying disease process in combination with NMN or a derivative or salt thereof, as well as compositions comprising them is also compatible with the administration of analgesics, NSAIDs, cortisone, and/or cortisone derivatives in order to treat eruptions.

The immunosuppressant may be selected from azathioprine, cyclophosphamide, chlorambucil, cyclosporine, methotrexate, and combinations thereof. Preferably, the immunosuppressant may be methotrexate or cyclosporine, more preferably methotrexate.

The immunomodulator may be selected from leflunomide, sulphasalazine, and combinations thereof.

Advantageously, the B-lymphocyte inhibitor may be rituximab. In particular, rituximab bonds to CD20 B-lymphocytes.

Advantageously, the T-lymphocyte inhibitor may be abatacept. In particular, abatacept bonds to T-lymphocytes expressing CD80 and CD86.

The synthetic antimalarial may be selected from chloroquine, hydroxychloroquine, and combinations thereof.

The anti-TNF agent may be selected from infliximab, etanercept, adalimumab, certolizumab, golimumab, and combinations thereof.

The enzymatic Janus kinase inhibitor may be tofacitinib.

The anti-interleukin agent may be selected from anti-interleukin 1, anti-interleukin 6, anti-interleukin 12, an interleukin 17 inhibitor, an interleukin 23 inhibitor, and combinations thereof. In particular, the anti-interleukin 1 may be anakinra. The anti-interleukin 6 may be tocilizumab.

The interleukin 12 inhibitor may be ustekinumab. The interleukin 17 inhibitor may be selected from ixekizumab and secukinumab. The interleukin 23 inhibitor may be selected from ustekinumab and guselkumab.

Preferably, NMN or a pharmaceutically acceptable derivative or salt thereof is not used in combination with a compound selected from folate, S-adenosyl-Lmethionine (SAM), astaxanthine, berberine, pterostilbene, resveratrol, metformin, vofloxacin, and combinations thereof.

### Compositions

Compositions according to the invention may comprise nicotinamide mononucleotide or a pharmaceutically acceptable derivative or salt thereof, and at least one pharmaceutically acceptable excipient in order to prevent and/or treat RA.

In the context of this invention, ‘excipient’ refers to any substance other than NMN in the composition that has no therapeutic effect. The excipient does not interact chemically with NMN or any other additional therapeutic.

The excipient may be selected from bulking agents, lubricants, flavourings, colourings, emulsifiers, compacting agents, gellants, plasticisers, surfactants, or combinations thereof.

Compositions according to the invention may be formulated with supports, excipients, and diluents that are suitable in themselves for these formulations, such as lactose, dextrose, saccharose, sorbitol, mannitol, starches, acacia gum, calcium phosphate, alginates, tragacanth gum, gelatin, calcium silicate, microcrystalline cellulose, polyvinylpyrrolidone, polyethylene glycol, cellulose, water (sterile), methylcellulose, methyl and propyl hydroxybenzoates, talc, magnesium stearates, edible oils, vegetable and mineral oils or suitable mixtures thereof. The formulations may optionally contain other substances commonly used in pharmaceutical formulations, such as lubricants, wetting agents, emulsifiers, suspending agents, dispersants, disintegrants, bulking agents, preservatives, sweeteners, flavourings, flow regulators, releasing agents, etc. The compositions may also be formulated for rapid, sustained, or delayed release of the active ingredient(s) they contain. Persons skilled in the art will know which excipients to select based on the galenic form selected.

The compositions according to the invention are preferably in unit dosage forms, and maybe suitably packaged, e.g. in boxes, blister packs, vials, bottles, sachets, ampoules, or any other support or receptacle suitable for a single dose or multiple doses (that can be properly labelled); optionally with one or more notices containing information on the product and/or instructions for use.

The composition according to the invention may be a pharmaceutical composition. In this case, the excipient is a pharmaceutically acceptable excipient as defined supra.

The composition according to the invention may also be a dietary supplement.

In a preferred embodiment, the composition according to the invention may further comprise at least one additional therapeutic as defined supra for use in the prevention and/or treatment of RA as described supra.

When the composition according to the invention comprises at least one additional therapeutic, the composition may be provided in a fixed unit dose form. ‘Fixed unit dose form’ refers to a composition comprising at least two active ingredients formulated in a single dosage form.

Preferably, the composition according to the invention does not comprise a compound selected from folate, S-adenosyl-Lmethionine (SAM), astaxanthine, berberine, pterostilbene, resveratrol, metformin, vofloxacin, and combinations thereof.

### NMN Derivatives

According to the invention, the NMN derivative may be selected from dihydronicotinamide mononucleotide (‘NMN—H’), the compound of formula (I):

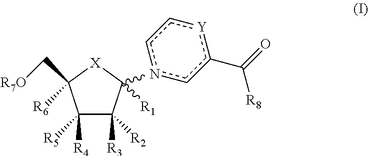

or a pharmaceutically acceptable stereoisomer, salt, hydrate, solvate, or crystal thereof, wherein
X is selected from O, CH₂, S, Se, CHF, CF₂, C═CH₂;
R₁ is selected from H, azido, cyano, C₁-C8 alkyl, C₁-C8 thioalkyl, C₁-C8 heteroalkyl, and OR; wherein R is selected from H and C₁-C8 alkyl;
R₂, R₃, R₄, and R₅ are selected independently of one another from H, halogen, azido, cyano, hydroxyl, C₁-C12 alkyl, C₁-C12 thioalkyl, C₁-C12 heteroalkyl, C₁-C12 haloalkyl, and OR; wherein R is selected from H, C₁-C₁₂ alkyl, C(O)(C₁-C₁₂) alkyl, C(O)NH(C₁-C₁₂) alkyl, C(O)O(C₁-C₁₂) alkyl, C(O) aryl, C(O)(C₁-C₁₂) alkylaryl, C(O)NH(C₁-C₁₂) alkylaryl, C(O)O(C₁-C₁₂) alkylaryl, and C(O)CHR_{AA}NH₂; wherein R_{AA} is a side chain selected from the proteinogenic amino acids;
R₆ is selected from H, azido, cyano, C₁-C8 alkyl, C₁-C8 thioalkyl, C₁-C8 heteroalkyl, and OR; wherein R is selected from H and C₁-C8 alkyl;
R₇ is selected from P(O)R₉R₁₀ and P(S)R₉R₁₀; wherein
R₉ and R₁₀ are selected independently of one another from OH, OR₁₁, NHR₁₃, NR₁₃R₁₄, C₁-C₈ alkyl, C₂-C₈ alkenyl, C₂-C₈ alkynyl, C₃-C₁₀ cycloalkyl, C₅-C12 aryl, (C₁-C₈) alkylaryl, (C₁-C₈) arylalkyl, (C₁-C₈) heteroalkyl, (C₁-C₈) heterocycloalkyl, heteroaryl, and NHCHR_{A}R_{A′}C(O)R₁₂; wherein:
R₁₁ is selected from a C₁-C10 alkyl, C₃-C10 cycloalkyl, C₅-C18 aryl, C₁-C10 alkylaryl, substituted C₅-C12 aryl, C₁-C10 heteroalkyl, C₃-C10 heterocycloalkyl, C₁-C₁₀ haloalkyl, heteroaryl, —(CH₂)ₙC(O)(C₁-C₁₅) alkyl, —(CH₂)ₙOC(O)(C₁-C₁₅) alkyl, —(CH₂)ₙOC(O)O(C₁-C₁₅) alkyl, —(CH₂)ₙSC(O)(C₁-C₁₅) alkyl, —(CH₂)ₙC(O)O(C₁-C₁₅) alkyl, and —(CH₂)ₙC(O)O(C₁-C₁₅)alkylaryl group; wherein n is an integer from 1-8; P(O)(OH)OP(O)(OH)₂. halogen, nitro, cyano, C₁-C₆ alkoxy, C₁-C₆ haloalkoxy, —N(R₁₁ₐ)₂, C₁-C₆ acylamino, —COR_{11b}, —O COR_{11b}; NHSO₂(C₁-C₆ alkyl), —SO₂N(R₁₁ₐ)₂SO₂; wherein each R₁₁ₐ is independently selected from H and C₁-C₆ alkyl, and R_{11b} is independently selected from OH, C₁-C₆ alkoxy, NH₂, NH(C₁-C₆ alkyl), and N(C₁-C₆ alkyl)₂;
R₁₂ is selected from H, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈ aryl, C₁-C₄ alkylaryl, and C₅-C₁₂ heteroaryl; wherein the aryl or heteroaryl groups are optionally substituted with one or two groups selected from halogen, trifluoromethyl, C₁-C₆ alkyl, C₁-C₆ alkoxy, and cyano; and
R_{A} and R_{A′} are independently selected from H, C₁-C10 alkyl, C₂-C10 alkenyl, C₂-C₁₀ alkynyl, C₃-C₁₀ cycloalkyl, C₁-C10 thioalkyl, C₁-C10 hydroxylalkyl, C₁-C10 alkylaryl, and C₅-C12 aryl, C₃-C10 heterocycloalkyl, heteroaryl, —(CH₂)₃NHC(═NH)NH₂, (1H-indol-3-yl) methyl, (1H-imidazol-4-yl) methyl, and a side chain selected from a proteinogenic or non-proteinogenic amino acid; wherein the aryl groups are optionally substituted with a group selected from hydroxyl, C₁-C10 alkyl, C₁-C₆ alkoxy, halogen, nitro, and cyano; or
R₉ and R₁₀ form, together with the phosphorus atoms to which they are attached, a 6-membered cycle, wherein —R₉-R₁₀— is —CH₂—CH₂—CHR—; wherein R is selected from a (C₅-C₆) aryl, and a (C₅-C₆) heteroaryl group; wherein the aryl or heteroaryl groups are optionally substituted with halogen, trifluoromethyl, C₁-C₆ alkyl, C₁-C6 alkoxy, and cyano; or
   R₉ and R₁₀ form, together with the phosphorus atoms to which they are attached, a 6-membered cycle, wherein —R₉—R₁₀— is —O—CH₂—CH₂—CHR—O—; wherein R is selected from a (C₅-C₆) aryl, and a (C₅-C₆) heteroaryl group; wherein the aryl or heteroaryl groups are optionally substituted with halogen, trifluoromethyl, (C₁-C₆) alkyl, (C₁-C6) alkoxy, and cyano;
R₈ is selected from H, OR, NHR₁₃, NR₁₃R₁₄, NH—NHR₁₃, SH, CN, N₃, and halogen; wherein R₁₃ and R₁₄ selected, independently of one another, from H, (C₁-C₈) alkyl, (C₁-C₈) alkylaryl, and —CR_{B}R_{C}—C(O)—OR_{D}; wherein R_{B} and R_{C} are independently a hydrogen atom, (C₁-C₆) alkyl, (C₁-C₆) alkoxy, benzyl, indolyl, or imidazolyl; wherein the (C₁-C₆) alkyl and the (C₁-C₆) may, optionally and independently of one another, be substituted by one or more halogen, amino, amido, guanidyl, hydroxyl, thiol, or carboxyl groups; and the benzyl group is optionally substituted by one or more halogen or hydroxyl groups; or R_{B} and R_{C}, together with the carbon atom to which they are attached, form a C₃-C₆ cycloalkyl group optionally substituted with one or more halogen, amino, amido, guanidyl, hydroxyl, thiol, and carboxyl; and RD is hydrogen, (C₁-C₆) alkyl, (C₂-C₆) alkenyl, (C₂-C₆) alkynyl, or (C₃-C₆) cycloalkyl;
Y is selected from CH, CH₂, C(CH₃)₂, and CCH₃;
[symbol] is a single or double bond depending on Y; and
[symbol] is the alpha or beta anomer depending on the position of R₁
or a pharmaceutically acceptable stereoisomer, salt, hydrate, solvate, or crystal thereof, or a combination thereof.

In a first preferred embodiment, the pharmaceutically acceptable derivative is the compound of formula (I).

In one variant of the first embodiment, X is oxygen.

In one variant of the first embodiment, R₁ and R₆ each, independently of one another, are hydrogen.

In one variant of the first embodiment, R₂, R₃, R₄, and R₅ each, independently of one another, are hydrogen or OH.

In one variant of the first embodiment, Y is CH.

In one variant of the first embodiment, Y is CH₂.

In one variant of the first embodiment, R₇ is P(O)(OH)₂.

In a variant of the first embodiment, the compound of the invention is selected from the following compounds:

TABLE 1

| Compound | |
|---|---|
| (anomer) | Structure |
| I-A (Beta) | 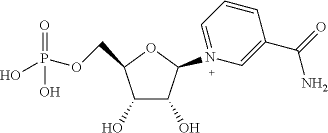 |
| I-B (beta) | 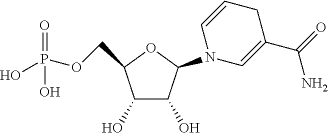 [SMILES: NC(=O)C1=CN([C@@H]2O[C@H](COP(=O)(O)O)[C@@H](O)[C@H]2O)C=CC1] |

The NMN derivative may be dihydronicotinamide mononucleotide (‘NMN—H’).

### Method for Preparing Compounds of Formula (I)

The derivatives of formula (I) may be prepared by any method well known to persons skilled in the art.

The derivatives of formula (I) may be prepared according to the method described in international application WO 2017/024255A1 or U.S. Pat. No. 10,611,790 B2.

In particular, the derivatives of formula (I) may be prepared according to the method described infra.

In particular, the compounds of formula (I) disclosed herein may be prepared as described infra from the substrates A-E. It will be understood by persons skilled in the art that these reaction schemes are by no means limiting, and that variations are possible without departing from the spirit and scope of this invention.

In one embodiment, the invention concerns a method for preparing compounds of formula (I) as described supra.

In a first step, the method includes monophosphorylating a compound of formula (A) in the presence of phosphoryl chloride and a trialkyl phosphate in order to afford the phosphorodichloridate of formula (B),

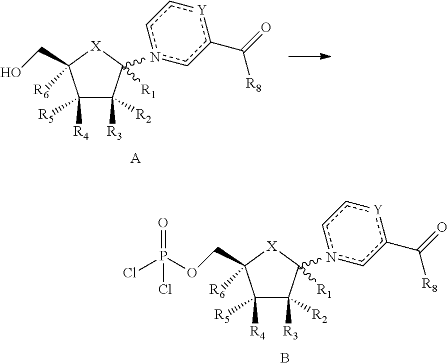

wherein X, R₁, R₂, R₃, R₄, R₅, R₆, R₈, Y, [symbol] , and [symbol] are as defined supra for compounds of formula (I).

In a second step, the phosphorodichloridate of formula (B) is hydrolysed, resulting in the phosphate of formula (C),

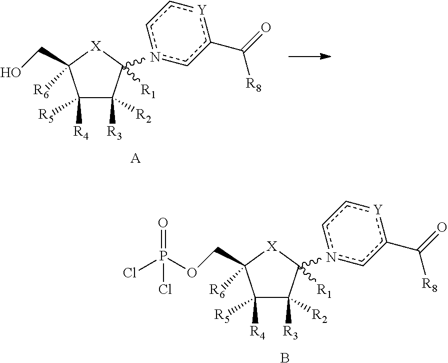

wherein X, R₁, R₂, R₃, R₄, R₅, R₆, R₈, Y, [symbol] , and [symbol] are as defined supra for compounds of formula (I).

In one embodiment, the compound of formula (A) is synthesised using various methods known to persons skilled in the art.

In one embodiment, the compound of formula (A) is synthesised by reacting the pentose of formula (D) with a nitrogenous derivative of formula (E), Wherein R, R₂, R₃, R₄, R₅, R₆, R₇, Y are as described supra for the compounds of formula I, resulting in the compound of formula (A-1), which is then selectively deprotected to afford the compound of formula (A),

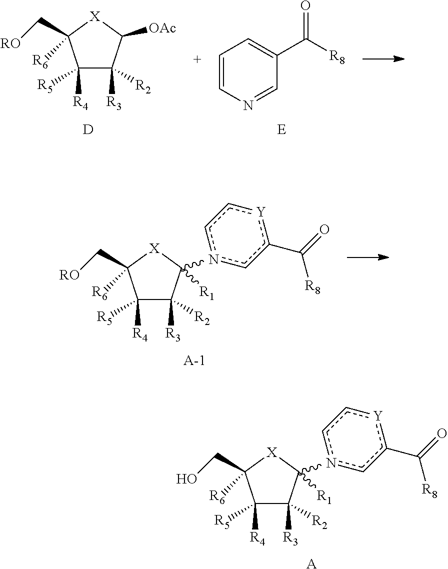

wherein X, R₁, R₂, R₃, R₄, R₅, R₆, R₈, Y, [symbol] , and [symbol] are as defined supra for compounds of formula (I).

In one embodiment, R is a suitable protecting group known to persons skilled in the art. In one embodiment, the protecting group is selected from triarylmethyls and/or silyls. Non-limiting examples of triarylmethyl include trityl, monomethoxytrityl, 4,4′-dimethoxytrityl, and 4,4′,4″-trimethoxytrityl groups. Non-limiting examples of silyl groups include trimethylsilyl, tert-butyldimethylsilyl, triisopropylsilyl, tert-butyldiphenylsilyl, tri-iso-propylsilyloxymethyl, and [2-(trimethylsilyl)ethoxy]methyl groups.

In one embodiment, any hydroxyl group attached to the pentose is protected by a suitable protecting group known to persons skilled in the art.

The selection and exchange of protecting groups is within the general knowledge of persons skilled in the art. Protecting groups may also be eliminated by methods well known to persons skilled in the art, e.g. with an acid (e.g. a mineral or organic acid), a base, or a fluoride source.

In a preferred embodiment, the nitrogenous derivative of formula (E) is coupled to the pentose of formula (D) by a reaction in the presence of a Lewis acid, resulting in the compound of formula (A-1). Non-limiting examples of Lewis acids include TMSOTf, BF₃.OEt₂, TiCl₄, and FeCl₃.

In one embodiment, the method of this invention further comprises a step of reducing the compound of formula (A) by various methods well known to persons skilled in the art, resulting in the compound of formula (A′), wherein [sic] is CH₂, and R₁, R₂, R₃, R₄, R₅, R₆, R₈, Y, [symbol] , and [symbol] are as defined supra for compounds of formula (I).

In a first step, the nicotinamide of formula E is coupled to the ribose tetraacetate of formula D by a reaction in the presence of a Lewis acid, resulting in the compound of formula A-1.

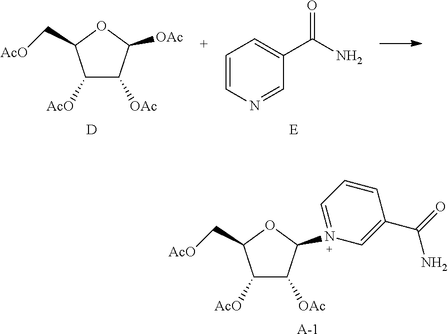

In a second step, the compound of formula A-1 is treated with ammonia, resulting in the compound of formula I-A:

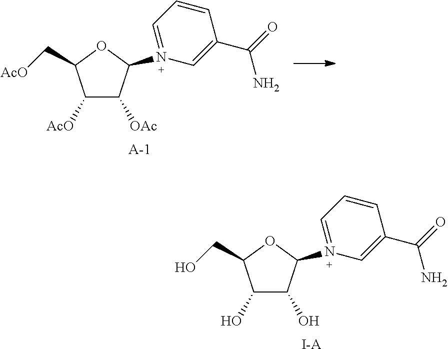

In a third step, monophosphorylation of the compound of formula I-A in the presence of phosphoryl chloride and a trialkyl phosphate results in the phosphorodichloridate of formula I-A′:

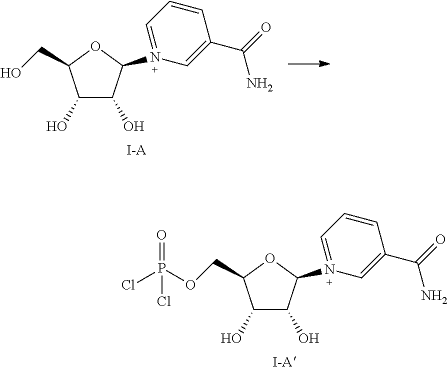

In a fourth step, the phosphorodichloridate of formula B is hydrolysed, resulting in the compound of formula I-C,

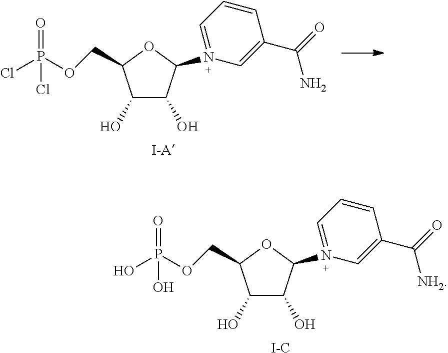

In one embodiment, a step of reducing the compound of formula I-A is carried out, resulting in the compound of formula I-E of the formula infra:

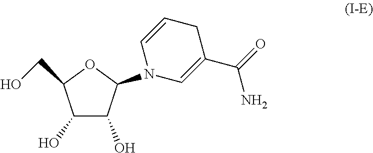 [SMILES: NC(=O)C1=CN([C@@H]2O[C@H](CO)[C@@H](O)[C@H]2O)C=CC1]

The compound of formula I-E is then monophosphorylated, as described for the fourth step, and hydrolysed, resulting in the compound of formula I-G.

In one embodiment, the compounds of formula (I) are selected from the compounds in the following table:

TABLE 1

| Compound | |
|---|---|
| (anomer) | Structure |
| I-A (Beta) | 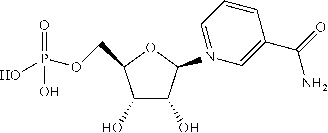 |
| I-B (beta) | 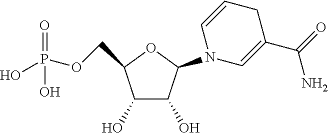 [SMILES: NC(=O)C1=CN([C@@H]2O[C@H](COP(=O)(O)O)[C@@H](O)[C@H]2O)C=CC1] |

In a preferred embodiment, the compounds of the invention are the compounds of the table supra or a pharmaceutically acceptable salt and/or solvate thereof.

### FIGURES

FIG. 1 is a graph of the development of the average weight of mice as a function of treatments.

FIG. 2 is a graph of the development of the average clinical score as a function of treatments.

FIG. 3 is a graph of the development of the average paw thickness of mice as a function of treatments.

FIG. 4 shows photographs of the paws of mice treated from each treatment group.

### EXAMPLE

In the following, the examples are provided solely to illustrate this invention, and by no means to limit its scope.

The efficacy of the use of NMN according to the invention was evaluated in mice in an RA model. In brief, 8-week-old female C57BL6/J mice, weighing between 18-20 g on day 0 of the experiments, were divided into 4 group of 10 mice each: (i) a control group, in which the mice were treated with vehicle, i.e. a 0.9% NaCl solution (10 mL/kg), marked ‘Vehicle’; (ii) a group of mice treated with K/BxN serum (10 mL/kg), marked ‘KBxN’; (iii) a group of mice treated with K/BxN serum+dexamethasone (1 mg/kg), marked ‘KBxN+dexamethasone’; and (iv) a group of mice treated with K/BxN serum+NMN (185 mg/kg), marked ‘KBxN+NMN’.

K/BxN serum is a serum obtained from genetically modified mice used as a model for RA. The administration of this serum induced chronic joint inflammation in mice in order to mimic RA. Dexamethasone is a cortisone derivative, a treatment conventionally used to combat RA eruptions. Here, NMN was used in zwitterionic form. KBxN serum and NMN were administered intraperitoneally to the nice. Dexamethasone was administered subcutaneously.

The mice were treated for 9 days under the conditions set forth supra. Photos of the paws of the mice were taken on the 6^{th} day of treatment. Blood samples were taken from the mice on day 6 and 10 of treatment. Tissue samples were taken on day 10 of treatment for histological analysis, i.e. the day after the end of treatment. The clinical score and weight of the mice were measured daily. The clinical score was determined by measuring the thickness of each of the front and rear paws of the mice, and adding the associated score according to table 1 infra:

TABLE 1

| Rear paw thickness X | Front paw thickness X | Score |
|---|---|---|
| X < 2.5 | X < 1.8 | 0 |
| 2.51 < X < 2.8 | 1.81 < X < 2.1 | 1 |
| 2.81 < X < 3.1 | 2.11 < X < 2.4 | 2 |
| 3.1 < X | 2.41 < X | 3 |

The statistical analysis of the results was carried out using two-factor ANOVA combined with the Dunnett multiple comparison test. The significance of the values is indicated as shown in table 2:

TABLE 2

| | | ‘KBxN + vehicle’ group |
|---|---|---|
| | ‘Vehicle’ group | compared to ‘KBxN + |
| | compared with | dexamethasone’ or |
| | ‘KBxN + vehicle’ group | ‘KBxN + NMN’ groups |
| p < 0.05 | + | * |
| p < 0.01 | ++ | ** |
| p < 0.001 | +++ | *** |
| p < 0.0001 | ++++ | **** |

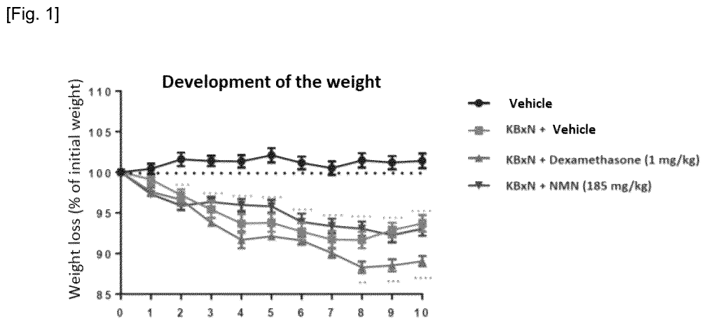

As can be seen in FIG. 1, the mice of the control group did not lose any weight over the duration of the experiment. On the other hand, the administration of K/BxN serum did induce significant weight loss in the three treatment groups, indicating treatment-related suffering on the part of the animals. The administration of dexamethasone induced additional and significant weight loss relative to the group treated with KBxN only, as well as the KBxN+NMN group. These observations are expected, and validate the experimental model. On the other hand, NMN did not cause significant weight loss relative to the KBxN group. As such, NMN was well tolerated and, more specifically, NMN was better tolerated than dexamethasone.

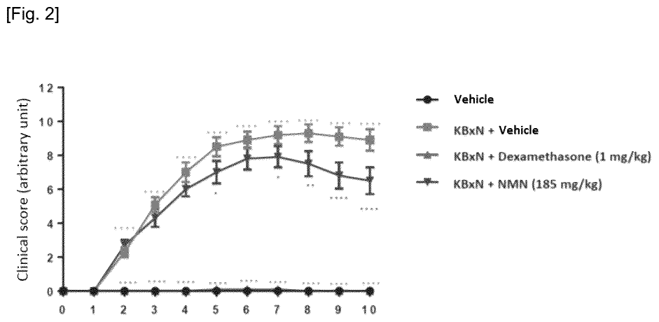

FIG. 2 shows the development of the clinical score amongst the various groups of mice. As can be seen in this figure, the mean clinical score of the mice of the control group is nil: thus, the vehicle does not cause any joint inflammation. Treatment of the mice with dexamethasone brings the clinical score to values near those of the control group. On the other hand, the clinical score of the mice treated with KBxN increased up to day 6, where it reached a plateau, indicating suffering and substantial inflammation in the mice. Treatment with NMN caused a significant reduction in clinical score starting on day 5. The reduction in clinical score became pronounced starting on day 8 of treatment. As such, the administration of NMN significantly reduces joint inflammation in this RA model.

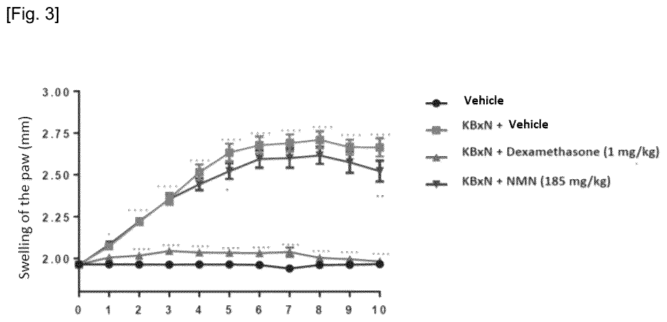

As shown in FIG. 3, the administration of KBxN induces paw swelling in the mice, consistent with the arthritis model. Treatment with dexamethasone significantly reduces ankle joint inflammation in the mice. Likewise, NMN treatment significantly reduced ankle joint inflammation starting on day 5, indicating the NMN reduces the joint inflammation induced by K/BxN serum.

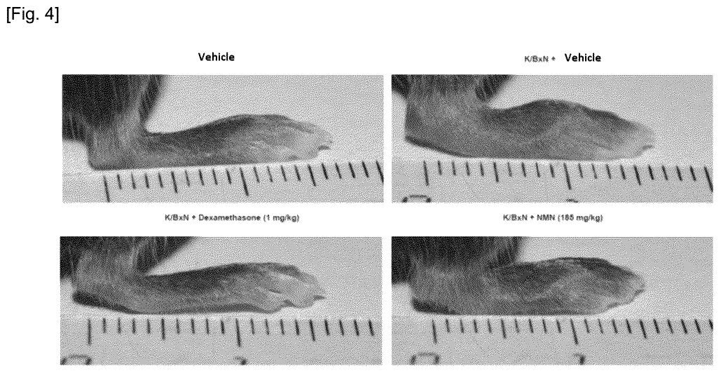

These observations were validated by the photographs of the paws of the mice shown in FIG. 4 that were taken on day 6, which show that the paw of the mouse treated with K/BxN is very swollen compared to the mouse from the control group, a sign of substantial joint inflammation. On the other hand, treatment with dexamethasone and NMN substantially reduces paw swelling, and, thus, joint inflammation. More precisely, as shown in the photographs in FIG. 4, the paw of the mouse treated with dexamethasone is much more slender than that of the mouse treated with vehicle, a sign of substantial weight loss on the part of the mouse. Although it is effective, dexamethasone causes suffering to the mouse due to its side-effects. On the other hand, the paw of the mouse treated with NMN has a volume similar to that of the mouse treated with vehicle. Thus, NMN is effective to treat the inflammation induced by K/BxN serum injection, and much better tolerated by the mice than conventional treatment with dexamethasone.

Thus, the administration of NMN significantly reduces the inflammation observed in an RA model without inducing the side-effects of dexamethasone, a cortisone derivative conventionally administered for the treatment of RA.

NMN or pharmaceutically acceptable derivatives or salts thereof, as well as compositions comprising them may thus be successfully and safely used to treat RA. Thus, this invention provides a therapeutic alternative to conventional treatments for RA, or at least proposes a therapeutic adjunct to conventional treatments in order to reduce their frequency and dosage. Due to the safety of NMN and its pharmaceutically acceptable derivatives and salts, as well as compositions comprising it, this invention allows for treatment and/or prevention of RA without inducing the side-effects caused by conventional treatments.

1-13. (canceled).

14. Nicotinamide mononucleotide (NMN), a pharmaceutically acceptable derivative, or a pharmaceutically acceptable salt thereof, for use in the prevention and/or treatment of rheumatoid arthritis.

15. Nicotinamide mononucleotide (NMN), a pharmaceutically acceptable derivative, or a pharmaceutically acceptable salt thereof, for use according to claim 14, wherein the pharmaceutically acceptable derivative of the NMN is dihydmnicotinamide mononucleotide (‘NMN—H’) the compound of formula (I): 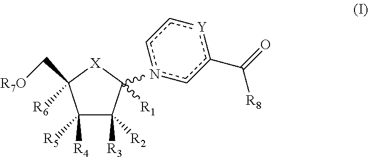
or a pharmaceutically acceptable stereoisomer, salt, hydrate, solvate, or crystal thereof, wherein
X is selected from O, CH₂, S, Se, CHF, CF₂, C═CH₂;
R₁ is selected from H, azido, cyano, (C₁-C₈) alkyl, (C₁-C₈) thioalkyl, (C₁-C₈) heteroalkyl, and OR: wherein R is selected from H and (C₁-C₈) alkyl;
R₂, R₃, R₄, and R₅ are selected independently of one another from H, halogen, azido, cyano, hydroxyl, (C₁-C₁₂) alkyl, (C₁-C₁₂) thioalkyl, (C₁-C₁₂) heteroalkyl (C₁-C₁₂) haloalkyl, and OR; wherein R is selected from H, (C₁-C₁₂) alkyl, C(O)(C₁-C₁₂) alkyl, C(O)NH(C₁-C₁₂) alkyl, C(O)O(C₁-C₁₂) alkyl, C(O) aryl, C(O)(C₁-C₁₂) alkylaryl, C(O)NH(C₁-C₁₂) alkylaryl, C(O)O(C₁-C₁₂) alkylaryl, and C(O)CHR_{AA}NH₂; wherein R_{AA} is a side chain selected from the proteinogenic amino acids;
R₆ is selected from H, azido, cyano, (C₁-C₈) alkyl, (C₁-C₈) thioalkyl, (C₁-C₈) heteroalkyl, OR; wherein R is selected from H and (C₁-C₈) alkyl;
R₇ is selected from P(O)R₉R₁₀ and P(S)R₉R₁₀; wherein
R₉ and R₁₀ are selected independently of one another from OH, OR₁₁, NR₁₃R₁₄, (C₁-C₈) alkyl, (C₂-C₈) alkenyl, (C₂-C₈) alkynyl, (C₃-C₁₀) cycloalkyl, (C₅-C₁₂) aryl, (C₁-C₈) alkylaryl, (C₁-C₈) arylalkyl, (C₁-C₈) heteroalkyl, (C₁-C₈) heterocycloalkyl, heteroaryl, and NHCHR_{A}R_{A′}C(O)R₁₂; wherein:
R₁₁ is selected from a (C₁-C₁₀) alkyl, (C₃-C₁₀) cycloalkyl, (C₅-C₁₈) aryl, (C₁-C₁₀) alkylaryl, substituted (C₅-C₁₂) aryl, (C₁-C₁₀) heteroalkyl, (C₃-C₁₀) heterocycloalkyl, (C₁C₁₀) haloalkyl, heteroaryl, —(CH₂)ₙC(O)(C₁-C₁₅) alkyl, —(CH₂)ₙOC(O)(C₁-C₁₅) alkyl, —(CH₂)ₙOC(O)O(C₁-C₁₅) alkyl, —(CH₂)ₙSC(O)(C₁-C₁₅) alkyl, —(CH₂)ₙC(O)O(C₁-C₅) alkyl, and —(CH₂)ₙC(O)O(C₁-C₁₅) alkylaryl group; wherein n is an integer from 1-8; P(O)(OH)OP(O)OH₂, halogen, nitro, cyano, C₁-C₆ alkoxy, C₁-C₆ haloalkoxy, —N(R₁₁ₐ)₂, C₁-C₆ acylamino, —COR_{11b}, —O COR_{11b}; NHSO₂(C₁-C₆ alkyl), —SO₂N(R₁₁ₐ)₂SO₂; wherein each R₁₁ₐ is independently selected from H and (C₁-C₆) alkyl, and R_{11b} is independently selected from OH, C₁-C₆ alkoxy, NH₂, NH(C₁-C₆ alkyl), and N(C₁-C₆ alkyl)₂;
R₁₂ is selected from H, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈ aryl, C₁-C₄ alkylaryl, and C₅-C₁₂ heteroaryl; wherein the aryl or heteroaryl groups are optionally substituted with one or two groups selected from halogen, trifluoromethyl. C₁-C₆ alkyl, C₁-C₆ alkoxy, and cyano; and
R_{A} and R_{A′} are independently selected from H (C₁-C₁₀) alkyl, (C₂-C₁₀) alkenyl, (C₂-C₁₀) alkynyl, (C₃-C₁₀) cycloalkyl, (C₁-C₁₀) thioalkyl, (C₁-C₁₀) hydroxylalkyl, (C₁-C₁₀) alkylaryl and (C₅-C₁₂) aryl, (C₃-C₁₀) heterocycloalkyl, heteroaryl, —(CH₂)₃NHC(═NH)NH₂, (1H-indol-3-yl) methyl, (1H-imidazol-4-yl) methyl, and a side chain selected from a proteinogenic or non-proteinogenic amino acid; wherein the aryl groups are optionally substituted with a group selected from hydroxyl, (C₁-C₁₀) alkyl, (C₁-C₆) alkoxy, un halogen, nitro, and cyano; or
R₉ and R₁₀ form, together with the phosphorus atoms to which they are attached, a 6-membered cycle, wherein is —R₉—R₁₀— is —CH₂—CH₂—CHR—; wherein R is selected from a (C₅-C₆) aryl, and a (C₅-C₆) heteroaryl group; wherein the aryl or heteroaryl groups are optionally substituted with halogen, trifluoromethyl, C₁-C₆ alkyl, (C₁-C₆) alkoxy, and cyano; or
R₉ and R₁₀ form, together with the phosphorus atoms to which they are attached, a 6-membered cycle, wherein —R₉—R₁₀— is —O—CH₂—CH₂—CHR—O—; wherein R is selected from a (C₅-C₆) aryl, and a (C₅-C₆) heteroaryl group; wherein the aryl or heteroaryl groups are optionally substituted with halogen, trifluoromethyl, (C₁-C₆) alkyl, (C₁-C6) alkoxy, and cyano;
R₈ is selected from H, OR, NHR₁₃, NR₁₃R₁₄, NH—NHR₁₃, SH, CN, N₃, and halogen. wherein R₁₃ and R₁₄ selected, independently of one another, from H, (C₁-C₈) alkyl, (C₁-C₈) alkylaryl, and —CR_{B}R_{C}—C(O)—OR_{D}; wherein R_{B} and R_{C} are independently a hydrogen atom, (C₁-C₆) alkyl, (C₁-C₆) alkoxy, benzyl, indolyl, or imidazolyl, wherein the (C₁-C₆) alkyl and the (C₁-C₆) may, optionally and independently of one another, be substituted by one or more halogen, amino, amido, guanidyl, hydroxyl, thiol, or carboxyl groups; and the benzyl group is optionally substituted by one or more halogen or hydroxyl groups; or R_{B} and R_{C}, together with the carbon atom to which they are attached, form a C₃-C₆ cycloalkyl group optionally substituted with one or more halogen, amino, amido, guanidyl, hydroxyl, thiol, and carboxyl; and R_{D} is hydrogen, (C₁-C₆) alkyl, (C₂-C₆) alkenyl, (C₂-C₆) alkynyl, or (C₃-C₆) cycloalkyl;
Y is selected from CH, CH₂, C(CH₃)₂, and CCH₃; [symbol]
is a single or double bond depending on Y; and [symbol]
is the alpha or beta anomer depending on the position of R₁
or a pharmaceutically acceptable stereoisomer, salt, hydrate, solvate, or crystal thereof, or a combination thereof.

16. Nicotinamide mononucleotide for use according to claim 14 in an amount between 0.01 mg/kg/d and 1000 mg/kg/d, preferably 1 mg/kg/d and 100 mg/kg/d, more preferably between 5 mg/kg/d and 50 mg/kg/d, even more preferably between 10 mg/kg/d and 20 mg/kg/d.

17. Nicotinamide mononucleotide for use according to claim 14, administered orally, intraocularly, sublingually, intravenously, intramuscularly, intraarticularly, subcutaneously, transcutaneously, vaginally, peridurally, intravesically, rectally, or by inhalation.

18. Nicotinamide mononucleotide for use according to claim 14, in combination with at least one other therapeutic.

19. Nicotinamide mononucleotide for use according to claim 18, wherein at the at least one therapeutic is an analgesic, an NSAID, cortisone, a cortisone derivative, an immunosuppressant, an immunomodulator, a T-lymphocyte inhibitor, a B-lymphocyte inhibitor, a synthetic antimalarial, an anti-TNF, an enzymatic Janus kinase inhibitor, an anti-interleukin, and combinations thereof.

20. Nicotinamide mononucleotide for use according to claim 19, wherein the at least one therapeutic is an immunosuppressant selected from methotrexate and cyclosporine, preferably methotrexate.

21. Composition comprising nicotinamide mononucleotide, a pharmaceutically acceptable derivative or salt thereof, and at least one pharmaceutically acceptable excipient, for use according to claim 20.

22. Composition for use according to claim 21, further comprising at least one additional therapeutic.

23. Composition for use according to claim 22, wherein at the at least one additional therapeutic selected from an NSAID, cortisone, a cortisone derivative, an immunosuppressant, immunomodulator, a T-lymphocyte inhibitor, a B-lymphocyte inhibitor, a synthetic antimalarial, an anti-TNF, an enzymatic Janus kinase inhibitor, an anti-interleukin, and combinations thereof.

24. Composition for use according to claim 23, provided as a fixed unit dose form.

25. Composition for use according to claim 22, wherein it is administered orally or by injection.

26. Composition for use according to claim 25, wherein it is administered in the form of a sublingual tablet or a gastroresistant capsule.

27. A method for the prevention and/or treatment of rheumatoid arthritis, comprising administering nicotinamide mononucleotide (NMN), a pharmaceutically acceptable derivative, or a pharmaceutically acceptable salt thereof to a person in need thereof.

28. The method of claim 27, wherein the pharmaceutically acceptable derivative of the NMN is dihydronicotinamide mononucleotide (‘NMN—H’), the compound of formula (I): 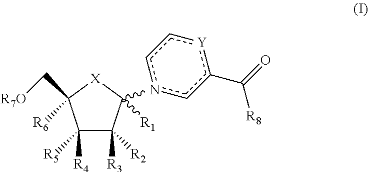
or a pharmaceutically acceptable stereoisomer, salt, hydrate, solvate, or crystal thereof, wherein
X is selected from O, CH₂, S, Se, CHF, CF₂, C═CH₂;
R₁ is selected from H, azido, cyano, (C₁-C₈) alkyl, (C₁-C₈) thioalkyl, (C₁-C₈) heteroalkyl, and OR; wherein R is selected from H and (C₁-C₈) alkyl;
R₂, R₃, R₄, and R₅ are selected independently of one another from H, halogen, azido, cyano, hydroxyl, (C₁-C₁₂) alkyl, (C₁-C₁₂) thioalkyl, (C₁-C₁₂) heteroalkyl, (C₁-C₁₂) haloalkyl, and OR; wherein R is selected from H, (C₁-C₁₂) alkyl, C(O)(C₁-C₁₂) alkyl, C(O)NH(C₁-C₁₂) alkyl, C(O)O(C₁-C₁₂) alkyl, C(O) aryl, C(O)(C₁-C₁₂) alkylaryl, C(O)NH(C₁-C₁₂) alkylaryl, C(O)O(C₁-C₁₂) alkylaryl, and C(O)CHR_{AA}NH₂; wherein R_{AA} is a side chain selected from the proteinogenic amino acids;
R₆ is selected from H, azido, cyano, (C₁-C₈) alkyl, (C₁-C₈) thioalkyl, (C₁-C₈) heteroalkyl, OR; wherein R is selected from H and (C₁-C₈) alkyl;
R₇ is selected from P(O)R₉R₁₀ and P(S)R₉R₁₀; wherein
R₉ and R₁₀ are selected independently of one another from OH, OR₁₁, NHR₁₃, NR₁₃R₁₄, (C₁-C₈) alkyl, (C₂-C₈) alkenyl, (C₂-C₈) alkynyl, (C₃-C₁₀) cycloalkyl, (C₅-C₁₂) aryl, (C₁-C₈) alkylaryl, (C₁-C₈) arylalkyl, (C₁-C₈) heteroalkyl, (C₁-C₈) heterocycloalkyl, heteroaryl, and NHCHR_{A}R_{A′}C (O)R₁₂; wherein:
R₁₁ is selected from a (C₁-C₁₀) alkyl, (C₃-C₁₀) cycloalkyl, (C₅-C₁₈) aryl, (C₁-C₁₀) alkylaryl substituted (C₅-C₁₂) aryl, (C₁-C₁₀) heteroalkyl, (C₃-C₁₀) heterocycloalkyl, (C₁-C₁₀ haloalkyl, heteroaryl, —(CH₂)ₙC(O)(C₁-C₁₅) alkyl, —(CH₂)ₙOC(O)(C₁-C₁₅) alkyl, —(CH₂)ₙOC(O)O(C₁-C₁₅) alkyl, —(CH₂)ₙSC(O)(C₁-C₁₅) alkyl, —(CH₂)ₙC(O)O(C₁-C₁₅) alkyl, and —(CH₂)ₙC(O)O(C₁-C₁₅) alkylaryl group; wherein n is an integer from 1-8; P(O)(OH)OP(O)(OH)₂. halogen, nitro, cyano, C₁-C₆ alkoxy, C₁-C₆ haloalkoxy, —N(R₁₁ₐ)₂, acylamino, —COR_{11b}, —O COR_{11b}; NHSO₂(C₁-C₆ alkyl), —SO₂N(R₁₁ₐ)₂SO₂; wherein each R₁₁ₐ is independently selected from H and (C₁-C₆) alkyl, and R_{11b} is independently selected from OH, C₁-C₆ alkoxy, NH₂, NH(C₁-C₆ alkyl), and N(C₁-C₆ alkyl)₂;
R₁₂ is selected from H, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈ aryl, C₁-C₄ alkylaryl, and C₅-C₁₂ heteroaryl; wherein the aryl or heteroaryl groups are optionally substituted with one or two groups selected from halogen, trifluoromethyl, C₁-C₆ alkyl, C₁-C₆ alkoxy, and cyano; and
R_{A} and R_{A′} are independently selected from H, (C₁-C₁₀) alkyl, (C₂-C₁₀) alkenyl, (C₂-C₁₀) alkynyl, (C₃-C₁₀) cycloalkyl, (C₁-C₁₀) thioalkyl, (C₁-C₁₀) hydroxylalkyl, (C₁-C₁₀) alkylaryl, and (C₅-C₁₂) aryl, (C₃-C₁₀) heterocycloalkyl, heteroaryl, —(CH₂)₃NHC(═NH)NH₂, (1H-indol-3-yl) methyl, (1H-imidazol-4-yl) methyl, and a side chain selected from a proteinogenic or non-proteinogenic amino acid; wherein the aryl groups are optionally substituted with a group selected from hydroxyl, (C₁-C₁₀) alkyl, (C₁-C₆) alkoxy, un halogen, nitro, and cyano; or
R₉ and R₁₀ form, together with the phosphorus atoms to which they are attached, a 6-membered cycle, wherein —R₉—R₁₀— is —CH₂—CH₂—CHR—; wherein R is selected from a (C₅-C₆) aryl, and a (C₅-C₆) heteroaryl group; wherein the aryl or heteroaryl groups are optionally substituted with halogen, trifluoromethyl, C₁-C₆ alkyl, (C₁-C₆) alkoxy, and cyano; or
R₉ and R₁₀ form, together with the phosphorus atoms to which they are attached, 6-membered cycle, wherein —R₉—R₁₀— is —O—CH₂—CH₂—CHR—O—; wherein R is selected from a (C₅-C₆) aryl, and a (C₅-C₆) heteroaryl group; wherein the aryl or heteroaryl groups are optionally substituted with halogen, trifluoromethyl, (C₁-C₆) alkyl, (C₁-C₆) alkoxy, and cyano;
R₈ is selected from H, OR, NHR₁₃, NR₁₃R₁₄, NH—NHR₁₃, SH, CN, N₃, and halogen. wherein R₁₃ and R₁₄ selected, independently of one another, from H, (C₁-C₈) alkyl, (C₁-C₈) alkylaryl, and —CR_{B}R_{C}—C(O)—OR_{D}, wherein R_{B} and R_{C} are independently a hydrogen atom, (C₁-C₆) alkyl, (C₁-C₆) alkoxy, benzyl, indolyl, or imidazolyl; wherein the (C₁-C₆) alkyl and the (C₁-C₆) may, optionally and independently of one another, be substituted by one or more halogen, amino, amido, guanidyl, hydroxyl, thiol, or carboxyl groups; and the benzyl group is optionally substituted by one or more halogen or hydroxyl groups; or R_{B} and R_{C}, together with the carbon atom to which they are attached, form a C₃-C₆ cycloalkyl group optionally substituted with one or more halogen, amino, amido, guanidyl, hydroxyl, thiol, and carboxyl; and R_{D} is hydrogen, (C₁-C₆) alkyl, (C₂-C₆) alkenyl, (C₂-C₆) alkynyl, or (C₃-C₆) cycloalkyl;
Y is selected from CH, CH₂, C(CH₃)₂, and CCH₃; [symbol]
is a single or double bond depending on Y; and [symbol]
is the alpha or beta anomer depending; on the position of R₁
or a pharmaceutically acceptable stereoisomer, salt, hydrate, solvate, or crystal thereof, or a combination thereof.
